(19) United States
(12) Patent Application Publication
(10) Pub. No.: US 2006/0287372 A1
(43) Pub. Date: Dec. 21, 2006

(54) Pharmaceutical preparations containing thiazolidinediones showing new therapeutic indications

(76) Inventors: Andrea Galli, Firenze (IT); Paola Romagnani, Firenze (IT); Mario Serio, Bagno A Ripoli (IT); Calogero Surrenti, Bagno A Ripoli (IT)

Correspondence Address: Jay S Cinamon;Abelman Frayne & Schwab, 666 Third Avenue 10th Floor, New York, NY, 10017-5621, US

(21) Appl. No.: 10/548,514

(22) PCT Filed: Mar. 2, 2004
(86) PCT No.: PCT/EP04/02069

§ 371 Date: Sep. 6, 2005

(30) Foreign Application Priority Data
Mar. 6, 2003 (IT) FI2003A000058

(51) Int. Cl. A61K 31/426

(52) U.S. Cl. 514/369

(57) ABSTRACT

The present invention refers to new therapeutic applications of pharmaceutical formulations containing, as active principles, thiazolidinediones.

### FIELD OF THE INVENTION

The present invention is connected to new therapeutic applications of thiazolidinediones also called glitazones, later on indicated as TzDs

### STATE OF ART

The TzDs represent a class of drug recently approved to improve glycemic control in patients affected by type 2 diabetes mellitus. The first of this class of drug, the ciglitazone, has been synthesized in 1982 (Fujita al 1983) and in spite of the study of this molecule has been interrupted, several compounds have been developed with different pharmacokinetics characteristics as Rosiglitazone and Pioglitazone, which are now commercially available.

A large body of experimental data seems to demonstrate that the majority of TzDs effects are due to interaction with PPARY.

The hypoglicemic effect of TzDs is due to the improvement of the peripherical action of insulin. They don't have any hypoglicemic effect in absence of insulin, in spite of the precise mechanism of action has not been completely elucidated. PPARγ is expressed in the adipose tissue and much less in liver, lung, colon and skeletal muscle. On the contrary, the hypoglicemic effect of TzDs seems due to the increase of glucose uptake in the skeletal muscle (80% of the glucose utilized by the body). The antagonizing activity of TzDs on the effects of TNF-α is able to determine an anti-inflammatory action. In the human macrophages TzDs are inhibiting the production of TNF-α as well as of other pro-inflammatory cytokines as IL-1β, IL-6 etc. In addiction through the interferences with the activity of the transcriptional factor NF-kB, AP 1 and STAT-1, the glitazones inhibit the expression of iNOS scavenger receptor A, gelatinase (MMP-9) and interleukin 8. Another important characteristics of the TzDs is the antiproliferative effect: the antitumoral action of these drugs has been observed in several tumours as liposarcoma breast, prostate, colon and thyroid cancer.

A large body of data seems to demonstrate important effects on endothelial cells. In rat models, troglitazone increases the NO levels stablizing mRNA of iNOS and decreases the expression of endothelis-1 and of inhibitor 1 of plasminogen activator which seems involved in atheroscherotic processes.

### DESCRIPTION OF THE FIGURES

FIG. 1 shows the decreasing production of IP-10 in primary cultures of stellate cells treated with Rosiglitazone and Pioglitazone

FIG. 2 shows the suppression of the IP-10 levels by treatment of mesangial cells with increasing doses of Rosiglitazone;

FIG. 3 shows the suppressive effects of Rosiglitazone on the IP-10 levels induced by interferon-γ in epithelial cells treated with increasing doses of Rosiglitazone.

In all the 3 figures the first column (0) indicates the IP-10 level after stimulation with INF-γ and TNF-α in the absence of TzDs.

### DETAILED DESCRIPTION OF THE INVENTION

It has been surprisingly found that in addiction to above mentioned pharmacological actions TzDs have other therapeutic never described activities, which cannot be supposed on the basis of previous literature.

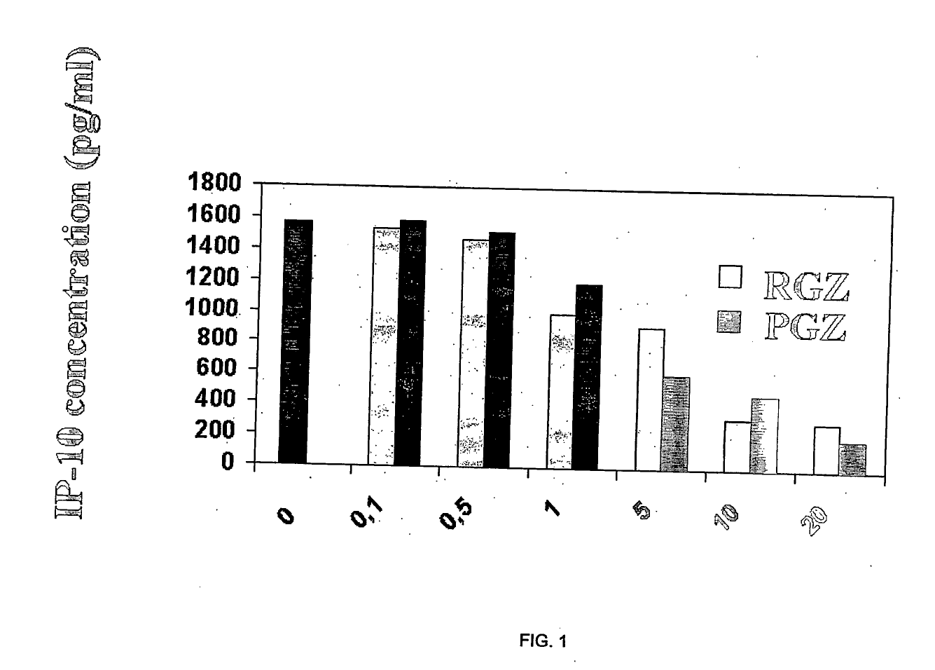

It has been demonstrated that the above mentioned drugs can have therapeutic effects on primary biliary cirrhosis, an autoimmune disease characterized by a progressive inflammatory damage of the liver, which induces a progressive fibrosis and cirrhosis. The biliary cirrhosis is characterized by high levels of IP-10 (CXCL 19) in blood which confirm the important role of this chemokine in the pathogenesis of the disease. In primary cultures of human stellate cells (Ito cells) we have demonstrate that TzDs are decreasing the production of IP-10 supporting the possible role of these drugs in the therapy of this disease. FIG. 1 shows the results of these experiments.

Another unsuspected field of application of interferon γ inducible chemokines is the possibility of their use as serum marker for prevision of immune susceptibility of a patient who undergoes to organ transplantation.

The parameter which today can be used to modulate the immuosuppressive therapy is the PRA (panel of reactive antibodies).

We found that the level of IP-10 (CXL-10) in transplanted patients allows the prevision of allographt rejection with high precision.

The IP-10 chemokine in fact plays an important role in the pathogenesis of acute and chronic rejection of allographt, as demonstrated by animal models. IP-10 plays a double biological role: it stimulates the migration of lymphocytes, macrophages, dendritic cells and other immunocompetent cells and regulates the vascular physiopathology by induction of mesangial expansion and inhibition of endothelial growth. These biological functions play a fundamental role in acute and chronic allographt rejection and represents together the. major cause of morbidity and of transplanted organ loss.

On the basis of the above mentioned experimental evidences, we based our hypothesis that an elevated pre-transplantation level of circulating IP-10 (due to chronic inflammatory stimolous by dyalis and uremia) can induce increased risk of allographt rejection. Therefore, we measured serum CXCL-10 levels before operation in 300 subjects undergoing kidney transplantation, then followed since 5 years from surgical intervention. The healthy controls were 50. In normal subjects the IP-10 levels were 84.4±29.9 pg/ml, while the levels of transplanted subjects were 137.6±123.2 pg/ml. IP-10 pre-transplantation levels were higher in the subjects who lost the allographt (130±116 pg/ml in non losers versus 200±163 pg/ml in losers). The subjects who lost the kidney during the firs year had pre-transplantation levels higher (211±165.1 vs 130.6±116 pg/ml). The survival curves according Kaplan-Meier, calculated in 300 patients divided in four groups (centiles) on the basis of IP-10 pre-transplantation levels, showed a progressive reduction of transplanted organ survival at 5 years according IP-10 values (97.3%, 94.0%, 93.3%, 85.3% of survival<0.05 in all groups; p<0.01 in the group 4 versus 1).

Between the group with the highest pre-transplantation levels of IP-10 and that with the lowest pre-transplantation levels a great difference of allographt loss has been observed (14.7% vs 2.7%, p<0.05). These differences were due to a major frequency of rejections in people with the highest IP-10 pre-transplantation levels. On these data is based the idea that the pre-transplantation levels of IP-10 can recognize the people with higher risk of undergoing an acute or chronic rejection and therefore of loosing the allographt. This people can be treated with more potent immuosuppressive therapy to avoid rejection.

On the basis of above reported considerations, it is evident that the presence in blood of IP-10 levels higher than those of healthy subjects it is an useful index to point out the rejection probability of an allographt. Consequently, the IP-10 levels represents a new diagnostic method for such purpose.

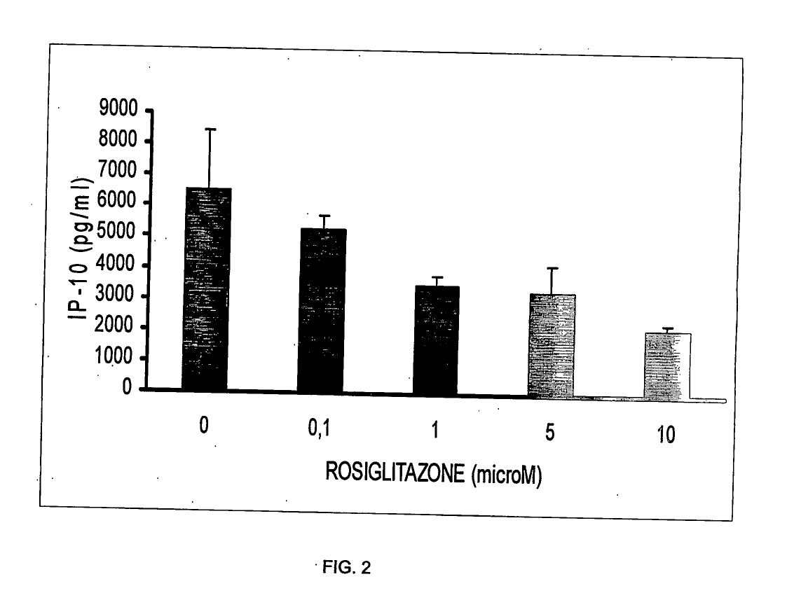

In addition, human mesangial cells in primary culture were treated by interferon γ (the physiological inducer of Mig ad IP-10 production) and with increasing dose of glitazones obtaining a dose dependent suppression of IP-10 (see FIG. 2).

Therefore, this is another object of present invention: the TzDs can be used in the prevention of acute and chronic rejection of kidney transplantation if they will be administered since the first days of transplantation to reduce the serum IP-10 levels and to blunt the inflammatory mechanism of lymphocyte recruitment which can induce an acute and/or chronic damage of allographt.

This effect seems particularly useful in relation to pancreatic islets transplantation, where we can combine the anti-rejection effect with the improvement of insulin resistance.

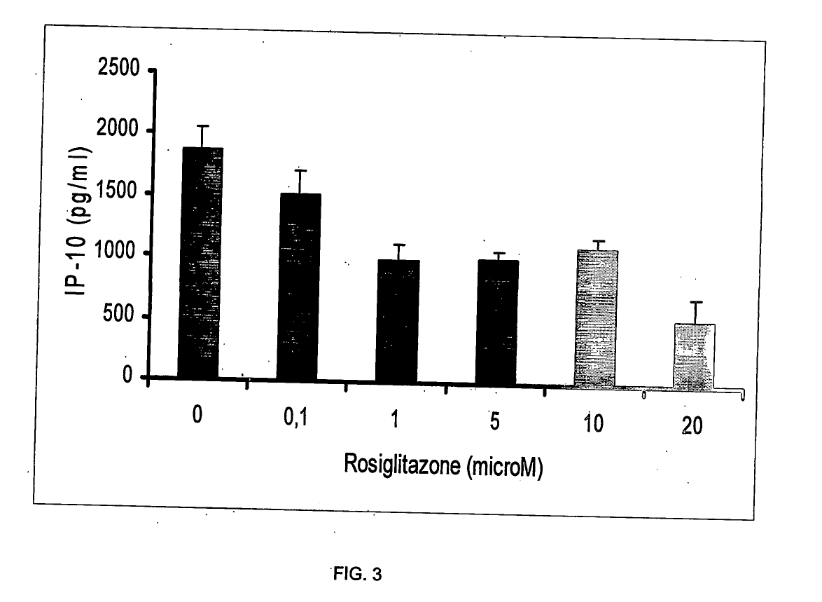

Lastly, the suppressive effects of glitazones on the IP-10 levels induced by interferon gamma have been also demonstrated by us in epithelial. cells (in particular thyrociytes) (see FIG. 3).

Since we have demonstrated that in recently onset Graves disease high circulating levels of IP-10 are present and that the interferon-γ induced chemokines are produced in thyroid as well as in other endocrine glands not only by inflammatory infiltrates but also by epithelia, glitazones can be considered drugs able to block the evolution of recent onset autoimmune endocrine diseases.

The formulation to be used according the present invention are similiar to those employed and commercialised usually as preparation and doses of active compound.

Also the daily doses can be comparable to those routinely used. For instance, formulation containing as active compound Rosiglitazone or Pioglitazone can be used as pills containing 15, 30, 45 mg of Piogliazone and 4 and 8 mg of Rosiglitazone.

1-8. (canceled)

9. A diagnostic method for the prevention of kidney transplantation rejection, which comprises measuring the IP-10 blood level of patients before transplantation according to known methods and comparing the measured levels to the known mean levels of healthy subjects (84.4±29.9 pg/ml).

10. A method for the prevention of acute and chronic rejection in kidney transplantation, which comprises administering thiazolidinediones to a kidney transplant patient beginning with the first day of transplantation to reduce the serum IP-10 levels.
